SEQUENCE LISTING (46 sequences)

SEQ ID NO: 1 - RNA, 22 nt, Homo sapiens
uagcuuaucagacugauguuga

SEQ ID NO: 2 - RNA, 24 nt, Homo sapiens
caaagugcuuacagugcagguagu

SEQ ID NO: 3 - RNA, 22 nt, Homo sapiens
uaaggugcaucuagugcaguua

SEQ ID NO: 4 - RNA, 23 nt, Homo sapiens
ugugcaaaucuaugcaaaacuga

SEQ ID NO: 5 - RNA, 23 nt, Homo sapiens
uaaagugcuuauagugcagguag

SEQ ID NO: 6 - RNA, 21 nt, Homo sapiens
uauugcacuugucccggccug

SEQ ID NO: 7 - RNA, 22 nt, Homo sapiens
uacccuguagauccgaauuugu

SEQ ID NO: 8 - RNA, 22 nt, Homo sapiens
uacccuguagaaccgaauuugu

SEQ ID NO: 9 - RNA, 22 nt, Homo sapiens
uuuggcacuagcacauuuuugc

SEQ ID NO: 10 - RNA, 22 nt, Homo sapiens
uuuggcaaugguagaacucaca

SEQ ID NO: 11 - RNA, 21 nt, Homo sapiens
ugguucuagacuugccaacua

SEQ ID NO: 12 - RNA, 23 nt, Homo sapiens
uauggcacugguagaauucacug

SEQ ID NO: 13 - RNA, 22 nt, Homo sapiens
uagcagcacauaaugguuugug

SEQ ID NO: 14 - RNA, 22 nt, Homo sapiens
uagcagcacaucaugguuuaca

SEQ ID NO: 15 - RNA, 22 nt, Homo sapiens
uagcagcacguaaauauuggcg

SEQ ID NO: 16 - RNA, 22 nt, Homo sapiens
ucccugagacccuaacuuguga

SEQ ID NO: 17 - RNA, 22 nt, Homo sapiens
uuaaggcacgcggugaaugcca

SEQ ID NO: 18 - RNA, 21 nt, Homo sapiens
uggaauguaaagaaguaugua

SEQ ID NO: 19 - RNA, 22 nt, Homo sapiens
uggaagacuagugauuuuguug

SEQ ID NO: 20 - RNA, 23 nt, Homo sapiens
agcagcauuguacagggcuauga

SEQ ID NO: 21 - RNA, 21 nt, Homo sapiens
ugugacugguugaccagaggg

SEQ ID NO: 22 - RNA, 22 nt, Homo sapiens
uauugcuuaagaauacgcguag

SEQ ID NO: 23 - RNA, 21 nt, Homo sapiens
ugcugacuccuaguccagggc

SEQ ID NO: 24 - RNA, 22 nt, Homo sapiens
caucuuaccggacagugcugga

SEQ ID NO: 25 - RNA, 23 nt, Homo sapiens
gcaaagcacacggccugcagaga

SEQ ID NO: 26 - RNA, 22 nt, Homo sapiens
agaggcuggccgugaugaauuc

SEQ ID NO: 27 - RNA, 22 nt, Homo sapiens
acuagacugaagcuccuugagg

SEQ ID NO: 28 - RNA, 22 nt, Homo sapiens
aagcugccaguugaagaacugu

SEQ ID NO: 29 - RNA, 23 nt, Homo sapiens
aacauucaacgcugucggugagu

SEQ ID NO: 30 - RNA, 21 nt, Homo sapiens
ugauuguccaaacgcaauucu

SEQ ID NO: 31 - RNA, 22 nt, Homo sapiens
uguaaacauccucgacuggaag

SEQ ID NO: 32 - RNA, 22 nt, Homo sapiens
ucacagugaaccggucucuuuu

SEQ ID NO: 33 - RNA, 21 nt, Homo sapiens
uagcaccaucugaaaucgguu

SEQ ID NO: 34 - RNA, 23 nt, Homo sapiens
uagcaccauuugaaaucaguguu

SEQ ID NO: 35 - RNA, 20 nt, Homo sapiens
uagcaccauuugaaaucggu

SEQ ID NO: 36 - RNA, 18 nt, Homo sapiens
ucuacagugcacgugucu

SEQ ID NO: 37 - RNA, 23 nt, Homo sapiens
uccagcaucagugauuuuguuga

SEQ ID NO: 38 - RNA, 22 nt, Homo sapiens
ccacugccccaggugcugcugg

SEQ ID NO: 39 - RNA, 23 nt, Homo sapiens
uauggcuuuuuauuccuauguga

SEQ ID NO: 40 - RNA, 21 nt, Homo sapiens
agggcccccccucaauccugu

SEQ ID NO: 41 - RNA, 22 nt, Homo sapiens
auauacauacacacaccuacac

SEQ ID NO: 42 - RNA, 22 nt, Homo sapiens
aacgcacuucccuuuagagugu

SEQ ID NO: 43 - RNA, 22 nt, Homo sapiens
uuaaugcuaaucgugauagggg

SEQ ID NO: 44 - RNA, 22 nt, Homo sapiens
uucaaguaauucaggauagguu

SEQ ID NO: 45 - RNA, 22 nt, Homo sapiens
uaacagucuacagccauggucg

SEQ ID NO: 46 - RNA, 21 nt, Homo sapiens
uaacagucuccagucacggcc

(19) United States
(12) Patent Application Publication
(10) Pub. No.: US 2010/0167948 A1
(43) Pub. Date: Jul. 1, 2010

(54) MicroRNA Expression Profiling of Cerebrospinal Fluid

(75) Inventors: Anna Krichevsky, Brookline, MA (US); Brit Mollenhauer, Brookline, MA (US)

Correspondence Address: LAW OFFICE OF MICHAEL A. SANZO, LLC, 15400 CALHOUN DR., SUITE 125, ROCKVILLE, MD, 20855, US

(73) Assignee: THE BRIGHAM AND WOMEN'S HOSPITAL, INC., Boston, MA (US)

(21) Appl. No.: 12/600,704

(22) PCT Filed: May 21, 2008
(86) PCT No.: PCT/US08/06452

§ 371 Date: Jan. 30, 2010

Related U.S. Application Data
(60) Provisional application No. 60/924,600, filed on May 22, 2007.

(51) Int. Cl. C40B 30/04; C12Q 1/68; C40B 40/06

(52) U.S. Cl. 506/9; 435/6; 506/16

(57) ABSTRACT

The present invention is directed to assay methods in which the levels of certain specific microRNAs are determined in the cerebrospinal fluid of a subject. These methods may be used in the diagnosis or monitoring of neurological diseases, especially brain tumors.

### CROSS REFERENCE TO RELATED APPLICATIONS

The present application claims priority to, and the benefit of, U.S. provisional application 60/924,600, filed on May 22, 2007, the contents of which is hereby incorporated by reference in its entirety.

### STATEMENT OF GOVERNMENT FUNDING

The United States Government has a paid-up license in this invention and the right in limited circumstances to require the patent owner to license others under reasonable terms as provided for by the terms of NIH grant R21CA116141, awarded by the Department of Health and Human Services.

### FIELD OF THE INVENTION

The present invention is directed to methods for diagnostically evaluating cerebrospinal fluid (CSF) based upon the relative amount of specific microRNAs that are present. These methods can be used in the detection and monitoring of neurological diseases.

### BACKGROUND OF THE INVENTION

MicroRNAs (miRNA) are small, single-stranded nucleic acids that bind to partly complementary sequences in mRNAs and thereby prevent them from being translated into protein. In this manner, miRNAs are believed to play a major role in regulating gene expression (Bartel, Cell 116:281-297 (2004); He, et al., Nat. Rev. Genet. 5:522-531(2004)). It has been found that miRNA levels are often altered in tumor cells and there have been suggestions that such alterations may contribute directly to the formation of some cancers (Meltzer, Nature 435:745-746 (2005)). As a result, there has been a great deal of interest in the diagnostic and therapeutic use of these molecules (see e.g., US 2007/0089196; 2005/0120415; 2005/0227934; 2005/02222067; 2006/0105360; 2006/0200878; 2006/0185025; and 2006/0019286).

Although, the diagnostic potential miRNA has been suggested, the use of cerebrospinal fluid (CSF) as a source for test samples has been largely overlooked. This is unfortunate since assays of CSF might be of considerable value in the diagnosis and monitoring of neurological diseases and, especially, brain tumors.

### SUMMARY OF THE INVENTION

General Summary

The present invention is based upon the discovery that some microRNAs are expressed at different levels in the normal and pathological brain and that these differences are reflected in the cerebrospinal fluid (CSF) of patients. These observations provide a basis for the concept that assays of CSF microRNA levels may be used in diagnosing brain diseases and in the post-therapy monitoring of patients. In particular, a comparison can be made between the levels of microRNA in the CSF of a test subject and in that of one or more control subjects. Comparisons can either be made directly or a comparison of the ratio of two microRNAs can be made. In the latter case, one would: a) measure the expression of two microRNAs; b) calculate the ratio of their expression levels; and c) determine the presence or progression of a disease if the ratio exceeds a certain threshold. For example, in the case of brain tumors, a ratio of miR-21 to mir-125b of greater than about 4 is indicative of a high grade glioma. Measurements of microRNA levels may be carried out using singleplex (involving one set of primers) or multiplex (involving more than one set of primers) qRT-PCR as described by, for example, Chen, et al. (Nucleic Acids Res. 33(20):e179 (2005), incorporated herein by reference in its entirety).

Specific Aspects of the Invention

In its first aspect, the invention is directed to a method of diagnosing or monitoring a neurological disease in a subject. The term “diagnosing” refers to the detection of disease in an individual that either has not previously had the disease or that has had the disease but who was treated and is believed to be cured. The term “monitoring” refers to tests performed on patients known to have a disease for the purpose of measuring its progress or for measuring the response of a patient to therapy. The method involves obtaining a test sample of cerebrospinal fluid (CSF) from the subject and assaying this sample to determine the concentration or amount of one or more microRNAs. The results obtained are compared with those obtained using control samples of CSF. The control samples may be from subjects known to be free of the disease or they may be from the general population. In cases where the method is being used to monitor a patient who has a disease or to test for the recurrence of a disease, the “control” sample may be test results obtained from the same patient at an earlier time, i.e., the patient may be examined for changes in microRNA levels before and after surgery or treatment.

It will be understood that it is not absolutely essential that an actual control sample be run at the same time that assays are being performed on a test sample. Once “normal,” i.e., control, levels of the microRNAs (or of microRNA ratios) have been established, these levels can provide a basis for comparison without the need to rerun a new control sample with each assay. The comparison between the test and control samples provides a basis for a conclusion as to whether a subject has a neurological disease (in cases where the method is being used diagnostically) or whether the disease is progressing or regressing in response to therapy (in cases where the method is being used for monitoring). In general, the greater the difference between the test sample and the control, the stronger the indication for the presence or progression of disease. At a minimum, a difference of 25% should be seen to conclude that a disease is present or progressing with higher differences (50%, 75%, 100% or more) being more conclusive.

The specific microRNAs that are tested for in the method discussed above include one or more of the following: miR-21; miR-17-5p; miR-18; miR-19; miR-20; miR-92; mir-10a; mir-10b; mir-96; mir-182/182*; mir-183; mir-15a; mir-15b; mir-16; mir-125b; mir-124; mir-1; mir-7; mir-103; mir-134; mir-137; mir-345; mir-200a; mir-330; mir-485-5p; mir-151; mir-22; mir-181; mir-219; mir-30; mir-128; mir-29a; mir-29b; mir-29c; mir-139; mir-338; mir-324-3p; mir-135; mir-296; mir-467; mir-521; and mir-155. The designations provided are standard in the art and are associated with specific sequences that can be found at the microRNA registry (http://microrna.sanger.ac.uk/sequences/). In all cases, they refer to human sequences as shown in Table 1. In some cases, there are additional family members of these microRNAs that are recognized in the art and which should be considered equivalents of the specific sequences listed herein. Although all sequences are shown as RNA sequences, it will be understood that, when referring to hybridizations or other assays, corresponding DNA sequences can be used as well. For example, RNA sequences may be reverse transcribed and amplified using the polymerase chain reaction (PCR) in order to facilitate detection. In these cases, it will actually be DNA and not RNA that is directly quantitated. It will also be understood that the complement of the reverse transcribed DNA sequences can be analyzed instead of the sequence itself. In this context, the term “complement” refers to an oligonucleotide that has an exactly complementary sequence, i.e. for each adenine there is a thymine, etc. Although assays may be performed for the microRNAs individually, it is generally preferable to assay several microRNAs or to compare the ratio of two of the microRNAs.

The microRNAs above will be particularly useful in the diagnosis and monitoring of cancers of the brain (e.g., gliomas, meningiomas, medulloblastomas, pituitary tumors, nerve sheath tumors, ependymomas, or CNS lymphomas). The most preferred of these microRNAs are miR-21 (increased levels being indicative of the presence or progression of a glioma) and mir-125b (decreased levels being indicative of the presence or progression of a glioma). The ratio of these two microRNAs may also be used to detect and monitor gliomas with a ratio of miR-21 to mir-125b of 3 or 4 being dispositive. Alternative microRNAs that may be used are mir-10b (increased in glioma) and mir-124 (decreased in glioma).

Specific neurological diseases that may be tested for using the methods described above include Alzheimer's disease; Huntington's disease; Parkinson's disease; amyotrophic lateral sclerosis; multiple sclerosis; stroke; and brain tumors. Of these, brain tumors are especially preferred with gliomas being the most preferred brain tumor. For Alzheimer's disease, the most preferred diagnostic microRNAs are mir-132 (decreased in AD), mir-212 (decreased in AD), mir-30a (increased in AD) and mir-26b (increased in AD).

In order to facilitate the testing of multiple microRNAs with the limited amounts of total RNA available from CSF, one of the following methods can be used: 1) multiplex and/or singleplex real-time RT-PCR (reagents available from, e.g., Applied Biosystems and System Biosciences (SBI)); 2) single-molecule detection (Neely, et al., Nat. Methods. 3(1):41-46 (2006); 3) bead-based flow cytometric methods (Lu, et al., Nature 435:7043 (2005); systems or reagents available from Luminex, Austin, Tex.); 4) array-based methods (e.g., Nelson, et al., Nat. Methods 1(2):155-61 (2004); Wu, et al., RNA 13(1): 151-159 (2007), all references being hereby incorporated by reference in their entirety). Microarrays can be prepared in which oligonucleotides having complementary sequences (or oligonucleotides with sequences matching the microRNAs themselves) are immobilized on the surface of a solid support. Materials that can be used as supports include membranes, and plates dishes or slides made of glass or plastic. At least 5 (and preferably, 10, 30 or more) of the microRNAs described above should be recognized by the immobilized oligonucleotides, with each different oligonucleotide occupying a distinct and known position on the support. Microarrays of this type may be made using methodology well known in the art or appropriate microRNA arrays can purchased commercially (e.g., from Ambion (Applied Biosystems), Foster City, Calif., Agilent or Exiqon). MicroRNA can then be isolated from the CSF (e.g., using Ambion's mirVana™ miRNA Isolation Kit) of a test subject, amplified using the polymerase chain reaction, and analyzed by hybridizations performed under stringent conditions.

### DETAILED DESCRIPTION OF THE INVENTION

The present invention is based upon the identification of microRNAs that are present in CSF and that can be used to identify patients with neurological diseases. These are shown in Table 1 and are all known in the art. References providing methods that can be used for amplifying and quantitating miRNA sequences include: Chen, et al., (Nucl. Ac. Res. 33(20):e179 (2005); Lao, et al., Biochem. Biophys. Res. Commun. 343(1):85-9 (2006); Lao, et al., Biotechnol. J. 2(1):33-5 (2007); Tang, et al., Nat. Protoc. 1(3):1154-1159 (2006); Neely, et al., Nat. Methods. 3(1):41-6 (2006) all incorporated by reference herein in their entirety).

Although an increased level of any of these microRNAs in the CSF of a subject is suggestive of the presence of disease, especially a brain tumor, a much better assessment can be made by examining many, preferably all, of the microRNAs. Many United States patents have issued describing techniques that can be used for detecting and quantitating microRNA and which may be used to analyze cerebrospinal fluid. These techniques include the following: detection by quantitative real time reverse transcriptase PCR (qRT-PCR) as described in patents owned by Applied Biosystems (U.S. Pat. Nos. 5,928,907; and 6,015,674), single-molecule detection as described in patents owned by US Genomics (U.S. Pat. Nos. 6,355,420; 6,916,661; and 6,632,526), bead-based assays as described in patents owned by Luminex (e.g., U.S. Pat. No. 6,524,793) and in assays using arrays of nucleic acids as described in patents owned by Ambion, Agilent, and Exiqon (U.S. Pat. Nos. 6,057,134; 6,891,032; 7,122,303; 6,458,583; 6,465,183; 6,461,816; 6,458,583; 7,026,124; 7,052,841; 7,060,809; 6,436,640; and 7,060,809). Other references providing guidance helpful in conducting assays include: patents generally describing techniques for producing microarray plates, slides and related instruments (U.S. Pat. No. 6,902,702; U.S. Pat. No. 6,594,432; U.S. Pat. Nos. 5,622,826; 5,556,752; 6,600,031; 6,576,424; 5,566,495; 6,551,784; and 6,887,655) and for carrying out assays (U.S. Pat. No. 6,902,900; U.S. Pat. No. 6,759,197). All of these patent references are hereby incorporated by reference herein in their entirety.

When microarray supports are used in assays they may be membranes or glass or plastic plates, slides or dishes having a series of distinct, immobilized oligonucleotides recognizing some or all of the microRNA sequences shown Table 1. The immobilized oligonucleotides must hybridize under stringent conditions to one of the microRNA sequences. The term “stringent conditions” indicates conditions that essentially only permit hybridization to occur with the exact complementary sequence of the immobilized oligonucleotide. In general, these hybridizations are performed in buffers of about neutral pH containing 0.1-0.5 NaCl and at a temperature of between 37-50° C. It is also possible to carry out incubations under conditions of low stringency and then to use high stringency wash conditions to cause the dissociation of hybridized sequences that are not exact matches.

One way to carry out microarray assays would involve amplifying microRNA in the presence of a detectable label, e.g., a nucleotide bound to a dye or other marker and present in a PCR primer. Thus, a population of labeled cDNAs may be obtained that can be used directly in hybridizations with oligonucleotides immobilized on a microarray plate or slide. After hybridizations are completed, plates may be analyzed using an automated reader to determine the amount of label associated with each immobilized sequence, which, in turn, reflects the abundance of the hybridized sequence in the original microRNA population. Many variations of this basic procedure have been described in the art and are compatible with the present invention.

TABLE 1
MicroRNA Sequences

| MicroRNA | Sequence | SEQ ID NO: |
|---|---|---|
| miR-21 | uagcuuaucagacugauguuga | 1 |
| miR-17-5p | caaagugcuuacagugcagguagu | 2 |
| miR-18b | uaaggugcaucuagugcaguua | 3 |
| miR-19 | ugugcaaaucuaugcaaaacuga | 4 |
| miR-20 | uaaagugcuuauagugcagguag | 5 |
| miR-92 | uauugcacuugucccggccug | 6 |
| mir-10a | uacccuguagauccgaauuugu | 7 |
| mir-10b | uacccuguagaaccgaauuugu | 8 |
| mir-96 | uuuggcacuagcacauuuuugc | 9 |
| mir-182 | uuuggcaaugguagaacucaca | 10 |
| mir-182* | ugguucuagacuugccaacua | 11 |
| mir-183 | uauggcacugguagaauucacug | 12 |
| mir-15a | uagcagcacauaaugguuugug | 13 |
| mir-15b | uagcagcacaucaugguuuaca | 14 |
| mir-16 | uagcagcacguaaauauuggcg | 15 |
| mir-125b | ucccugagacccuaacuuguga | 16 |
| mir-124 | uuaaggcacgcggugaaugcca | 17 |
| mir-1 | uggaauguaaagaaguaugua | 18 |
| mir-7 | uggaagacuagugauuuuguug | 19 |
| mir-103 | agcagcauuguacagggcuauga | 20 |
| mir-134 | ugugacugguugaccagaggg | 21 |
| mir-137 | uauugcuuaagaauacgcguag | 22 |
| mir-345 | ugcugacuccuaguccagggc | 23 |
| mir-200a | caucuuaccggacagugcugga | 24 |
| mir-330 | gcaaagcacacggccugcagaga | 25 |
| mir-485-5p | agaggcuggccgugaugaauuc | 26 |
| mir-151 | acuagacugaagcuccuugagg | 27 |
| mir-22 | aagcugccaguugaagaacugu | 28 |
| mir-181 | aacauucaacgcugucggugagu | 29 |
| mir-219 | ugauuguccaaacgcaauucu | 30 |
| mir-30a | uguaaacauccucgacuggaag | 31 |
| mir-128 | ucacagugaaccggucucuuuu | 32 |
| mir-29a | uagcaccaucugaaaucgguu | 33 |
| mir-29b | uagcaccauuugaaaucaguguu | 34 |
| mir-29c | uagcaccauuugaaaucggu | 35 |
| mir-139 | ucuacagugcacgugucu | 36 |
| mir-338 | uccagcaucagugauuuuguuga | 37 |
| mir-324-3p | ccacugccccaggugcugcugg | 38 |
| mir-135 | uauggcuuuuuauuccuauguga | 39 |
| mir-296 | agggcccccccucaauccugu | 40 |
| mir-467 | auauacauacacacaccuacac | 41 |
| mir-521 | aacgcacuucccuuuagagugu | 42 |
| mir-155 | uuaaugcuaaucgugauagggg | 43 |
| mir-26b | uucaaguaauucaggauagguu | 44 |
| mir-132 | uaacagucuacagccauggucg | 45 |
| mir-212 | uaacagucuccagucacggcc | 46 |

All references cited herein are fully incorporated by reference in their entirety. Having now fully described the invention, it will be understood by those of skill in the art that the invention may be practiced within a wide and equivalent range of conditions, parameters and the like, without affecting the spirit or scope of the invention or any embodiment thereof.

1. A method of diagnosing or monitoring a neurological disease in a subject, comprising:
a) obtaining a test sample of cerebrospinal fluid (CSF) from said subject;
b) assaying said test sample to determine the concentration or amount of one or more microRNAs;
c) comparing the concentration or amount determined in step b) with the concentration or amount determined for the same one or more microRNAs in one or more control samples of CSF; and
d) concluding that said subject has said neurological disease, or that said neurological disease has progressed or recurred, if the concentration or amount of said microRNA is at least 25% higher or lower in said test sample than in said one or more control samples.

2. The method of claim 1, wherein it is concluded in step d) that said subject has said neurological disease, or that said neurological disease has progressed or recurred, if the concentration or amount of said microRNA is at least 50% higher or lower in said test sample than in said one or more controls samples.

3. (canceled)

4. The method of claim 1, wherein said microRNA is selected from the group consisting of: miR-21; miR-17-5p; miR-18; miR-19; miR-20; miR-92; mir-10a; mir-10b; mir-96; mir-182/182*; mir-183; mir-15a; mir-15b; mir-16; mir-125b; mir-124; mir-1; mir-7; mir-103; mir-134; mir-137; mir-345; mir-200a; mir-330; mir-485-5p; mir-151; mir-22; mir-181; mir-219; mir-30a; mir-128; mir-29a; mir-29b; mir-29c; mir-139; mir-338; mir-324-3p; mir-135; mir-296; mir-467; mir-521; mir-155; mir-26b; mir-132; and mir-212.

5. The assay of claim 1, wherein said microRNA is mir-21or mir-10b and the presence or progression of said neurological disease is based upon the concentration or amount of said microRNA being increased by at least 25% relative to the concentration or amount present in said control sample.

6. The assay of claim 5, wherein said neurological disease is a cancer of the brain.

7. (canceled)

8. The assay of claim 1, wherein said microRNA is mir-125b or mir-124 and the presence or progression of said neurological disease is based upon the concentration or amount of said microRNA being decreased by at least 25% relative to the concentration or amount present in said control sample.

9. The assay of claim 8, wherein said neurological disease is a cancer of the brain.

10. (canceled)

11. The assay of claim 1, wherein said microRNA is mir-30a or mir-26b and the presence or progression of said neurological disease is based upon the concentration or amount of said microRNA being increased by at least 25% relative to the concentration or amount present in said control sample.

12. The assay of claim 11, wherein said neurological disease is Alzheimer's disease.

13. The assay of claim 1, wherein said microRNA is mir-132 or mir-212 and the presence or progression of said neurological disease is based upon the concentration or amount of said microRNA being decreased by at least 25% relative to the concentration or amount present in said control sample.

14. The assay of claim 13, wherein said neurological disease is Alzheimer's disease.

15. The method of claim 1, wherein said method is used to monitor a patient that has been treated for a neurological disease by surgery, radiation or medication and wherein said control sample is a CSF sample taken from said patient at an earlier time.

16-22. (canceled)

23. A microarray useful for diagnosing neurological diseases comprising:
a) a solid support comprising a membrane, glass or plastic dish, plate or slide; and
b) at least 5 distinct polynucleotides, each of which is attached to said solid support at a separate site and each of which hybridizes under stringent conditions to a different microRNA or complement thereof, wherein said microRNA is selected from the group consisting of: miR-21; miR-17-5p; miR-18; miR-19; miR-20; miR-92; mir-10a; mir10b; mir-96; mir-182/182*; mir-83; mir-15 a; mir-15b; mir-16; mir-125b; mir-124; mir-1; mir-7; mir-103; mir-134; mir-137; mir-345; mir-200a; mir-330; mir-485-5p ; mir-151; mir-22; mir-181; mir-219; mir-30; mir-128; mir-29a; mir-29b; mir-29c; mir-139; mir-338; mir-324-3p; mir-135; mir-296; mir-467; mir-521; mir-155; mir-26b; mir-132; and mir-212.

24. (canceled)

25. The microarray of claim 23, wherein said microarray includes distinct polynucleotides, hybridizing to at least 20 of said microRNAs or complements thereof

26. The microarray of claim 23, wherein said microarray includes distinct polynucleotides, hybridizing to at least 40 of said microRNAs or complements thereof

27. The microarray of claim 23, wherein said microarray includes distinct polynucleotides, hybridizing to 5-45 of said microRNAs or complements thereof.

28. (canceled)

29. The microarray of claim 23, wherein said microarray includes no more than 20 distinct polynucleotides, hybridizing to said microRNAs or complements thereof.

30. The microarray of claim 23, wherein said microRNAs include miR-21 and mir-10b.

31. The microarray of claim 30, wherein said microRNAs include miR-125b and mir-124.

32. An diagnostic assay for cancer of the brain comprising determining the amount or concentration of at least 5 distinct microRNAs in cerebrospinal fluid from a subject by performing a hybridization with the micrarray microarray of claim 23.
